(19) United States
(12) Patent Application Publication
(10) Pub. No.: US 2015/0233925 A1
(43) Pub. Date: Aug. 20, 2015

(54) Assays and Cell-Based Tests Using a Receptor Na/K-ATPase/Src Complex and Uses Thereof

(71) Applicant: The University of Toledo, Toledo, OH (US)

(72) Inventors: Zi-Jian Xie, Toledo, OH (US); Joseph I. Shapiro, Toledo, OH (US); Fangfang Lai, Toledo, OH (US)

(73) Assignee: THE UNIVERSITY OF TOLEDO, Toledo, OH (US)

(21) Appl. No.: 14/399,625

(22) PCT Filed: May 8, 2013
(86) PCT No.: PCT/US2013/040181

§ 371 Date: Nov. 7, 2014

Related U.S. Application Data
(60) Provisional application No. 61/644,232, filed on May 8, 2012.

(51) Int. Cl. G01N 33/573; A61K 36/232; A61K 36/481; A61K 36/537

(52) U.S. Cl. CPC G01N 33/573; A61K 36/537; A61K 36/232; A61K 36/481; G01N 2333/912; G01N 2500/02; G01N 2500/10; G01N 2500/20

(57) ABSTRACT

Described herein are assays and complementary cell culture based tests, and uses thereof for determining agonists or antagonists of Na/K-ATPase/Src complex, and methods of treatment therewith.

### CROSS-REFERENCE TO RELATED APPLICATIONS

This application claims the benefit of U.S. Provisional Application Ser. No. 61/664,232 filed in May 8, 2012, the entire disclosure of which is expressly incorporated herein by reference.

### STATEMENT REGARDING FEDERALLY SPONSORED RESEARCH

The invention was made with U.S. Government support under Grant Number HL-109015 awarded by the National Institutes of Health, Grant Number. The United States Government has certain rights in the invention.

### TECHNICAL FIELD AND INDUSTRIAL APPLICABILITY OF THE INVENTION

This invention relates to a protocol that screens agonists and antagonists of the receptor Na/K-ATPase/Src, and the identification of unsaturated fatty acids, glutathione disulfide (GSSG) and organosulfur compound derivatives as a partial agonist, and MB-5, curcumin, curcumin derivatives, Tanshensu, Tanshinone, Tanshinone derivatives, astragaloside IV and ferulic acid as the receptor antagonists.

### BACKGROUND OF THE INVENTION

Cancer is a leading cause of death across the globe. According to the World Health Organization (WHO), cancer accounted for 7.6 million deaths—about 13 percent of all deaths—worldwide in 2008, with that number expected to grow to 12 million deaths by 2030. The incidence of Congestive Heart Failure (CHF) is also on the rise, affecting five million people in the United States. Approximately 8 out of 1,000 people over age 70 are diagnosed with CHF.

Development of agonists of receptor Na/K-ATPase/Src complex is useful because physiological stimulation by agonists is essential for normal renal, cardiac and lung functions, and they protect organs. Development of antagonists is useful as therapeutics for cancer and chronic organ degeneration diseases because sustained-stimulation leads to abnormal growth (tumor) and organ fibrosis.

Na/K-ATPase has a role in balancing electrolyte and fluid in cells, organs and the whole body. The mammalian cell Na/K-ATPase interacts with various membranes and cytosolic proteins, thus serving as a signaling receptor. Src, a non-receptor tyrosine kinase can form a receptor complex with the Na/K-ATPase and regulate cell growth, differentiation, apoptosis, and fibrosis, as schematically illustrated in FIG. 1.

Activation of the Src receptor complex by physiological concentrations of cardiotonic steroids plays important roles in the regulation of renal, cardiac and vascular functions. Moreover, sustained activation of the Src receptor complex results in ROS stress that contributes to many diseases including cancer, hypertension, preeclampsia, end stage renal disease, congestive heart failure, and diabetes mellitus.

The Na/K-ATPase enzyme is ubiquitously expressed in most eukaryotic cells and is essential for maintaining the trans-membrane ion gradient by pumping Na⁺ out and K⁺ into cells. Structurally, the enzyme consists of two non-covalently linked α and β subunits. The Na/K-ATPase α subunit has 10 transmembrane domains with both N- and C-termini located in the cytoplasm. The α subunit consists of several well-characterized domains: actuator (A) domain consists of the N-terminus and the second cytosolic domain (CD2) connected to transmembrane helices M2 and M3; highly conserved discontinuous phosphorylation (P) domain is close to the plasma membrane; and a relatively isolated nucleotide-binding (N) domain. These structures also show a significant movement of A and N domain during the ion pumping cycle. The A domain rotates while the N domain closes up during the transport cycle, which opens (E1) and closes (E2) the A, N and P domains.

Previously, the inventors and others have demonstrated that binding of cardiotonic steroids (CTS) such as ouabain to the Na/K-ATPase stimulates multiple protein kinase cascades. Moreover, knockout of Src abolishes most of these activations. Src, a member of Src family non-receptor kinases, plays an important role in the signal transduction pathways of many extracellular stimuli, i.e., cytokines, growth factors and stress responses, and is a promising target for therapeutic interventions in certain cancers and bone diseases.

The Na/K-ATPase interacts directly with Src via at least two binding motifs: one being between the CD2 of the α1 subunit and Src SH2; and, other involving the third cytosolic domain (CD3) and Src kinase domain. The formation of this Na/K-ATPase and Src complex serves as a receptor, thus provoking protein kinase cascades.

For example, binding of ouabain to Na/K-ATPase will disrupt the latter interaction, and then result in assembly and activation of different pathways including ERK cascades, PLC/PKC pathway and mitochondrial ROS production. Moreover, this interaction keeps Src in an inactive state. Thus, the Na/K-ATPase functions as an endogenous negative Src regulator. See for example, the co-inventors' pending applications: US Pub. No. 2011/0245167 published Oct. 6, 2011, PCT/US07/023,011, filed Oct. 17, 2007 (Pub. No. WO 2808/054792 on May 8, 2008), claiming priority from U.S. Ser. No. 60/855,482 filed Oct. 16, 2006, which are expressly incorporated herein by reference.

There is a need for targeting the newly discovered Na/K-ATPase/Src receptor complex to develop novel agonists or antagonists of the receptor so that the receptor function of Na/K-ATPase/Src complex can be either stimulated for treating diseases such as ischemia/reperfusion injury or inhibited for treating diseases such as tissue fibrosis, congestive heart failure, and cancer.

Such a general method would be of tremendous utility in that whole families of related proteins each with its own version of the functional domain of interest could be identified. Knowledge of such related proteins would contribute greatly to our understanding of various physiological processes, including cell growth or death, malignancy, renal/cardiovascular function and immune reactions.

Such a method would also contribute to the development of increasingly more effective therapeutic, diagnostic, or prophylactic agents having fewer side effects. This is of increasing importance since, currently, people believe that the use of traditional Chinese medicine (TCM) to treat patients is both a gentle therapy and highly acceptable in commerce. At one time, plants and extracts thereof were the most significant group of substances used by healers to treat patients. Many of the world's population still rely heavily on herbal medicine as their primary source of therapy. Also, many of the active components of currently prescribed drugs were first identified in plants and many of the most popular drugs today are derived from plant materials.

In addition, such alternative approaches to medicine are becoming more and more widely accepted and used in the United States as well to treat a broad spectrum of conditions as well as to maintain wellness. It is estimated that one in two Americans currently uses alternative therapies at one time or another. In particular, the most popular complementary or fully alternative approach to the treatment of their cancers by patients is botanical agents/herbal medicines.

While such administration of the plant or an extract thereof to human patients has proceeded for centuries, there is still a great uncertainty as to the precise amounts which will provide an effective amount. That is, by the very nature of plant being a living form, each plant is unique in that one plant may vary in the amount of an effective ingredient therein. Also, different varies will contain different amounts of effective ingredients; for example, it has been noted that the more tropical the climate in which the plant originates, the more pungent the fruit.

In particular, traditional Chinese medicine (TCM) is often the treatment modality of choice by patients opting for an additional and/or alternative approach to dealing with their disorders. For Example, patients use TCM both as anti-cancer agents and to alleviate the side effects of standard chemotherapy. However, TCM lacks the scientifically sound methodology required of Western pharmacology and the use of TCM is often hit or miss in its effectiveness.

There remains a need for the discovery of specific herbal extracts and combinations thereof that have a specific utility and for which there is scientific evidence as to why they work in that use.

According to the present invention, just such novel compositions and methods are provided.

### SUMMARY OF THE INVENTION

The Na/K-ATPase α1 subunit and Src can form a receptor complex. Endogenous cardiotonic steroids (CTS) and digitalis drugs such as ouabain act as agonists and provoke this receptor complex, as shown in FIG. 1. Subsequently, this initiates downstream protein kinase cascades. The activation of these pathways through the receptor Na/K-ATPase/Src complex plays an important role in the regulation of cell growth, renal salt handling and organ remodeling.

Described herein is a method of identifying a compound that alters Src activity, where the method comprises:
i) purifying α1 Na/K-ATPase to obtain Na/K-ATPase exhibiting specific activity higher than 800 μmol Pi/mg protein/h;
ii) mixing the purified Na/K-ATPase of step (i) with Src to form a receptor Na/K-ATPase/Src complex having inhibited Src activity;
iii) exposing the receptor Na/K-ATPase/Src complex of step (ii) to a compound, wherein binding of the compound releases the inhibited Src from the receptor Na/K-ATPase/Src complex, resulting in an increase in Src activity; and,
iv) measuring the increase in Src activity, wherein the increased Src activity is indicative of the compound altering Src activity.

In certain embodiments, the method includes determining whether there is an enhanced level of Src activity as compared to a control level of Src activity, wherein enhancement in the level of Src activity indicates that the compound is an agonist, and, wherein a decrease in the level of Src activity relative to the control level indicates that the compound is an antagonist.

In certain embodiments, the agonist of the receptor Na/K-ATPase/Src complex comprises cardiotonic steroids.

In certain embodiments, the agonist of receptor Na/K-ATPase/Src complex comprises, comprising at least one or more of: ouabain, digoxin, marinobufagenin (MBG), oleic acid, docosahexaenoic (DHA), glutathione disulfide (GSSG) and allyl isothiocyanate.

In certain embodiments, the antagonist of receptor Na/K-ATPase/Src complex, comprises two or more of: 3,4,5-trihydroxyxanthone (MB5), 3,4,5,6-tetrahydroxyxanthone (MB7), curcumin, bisdemethoxycurcumin, tanshinone I, sodium danshensu, astragaloside IV, ferulic acid and tanshinone IIA.

In certain embodiments, the antagonist of receptor Na/K-ATPase/Src complex, comprises a combination of: tanshinone I, sodium danshensu, astragaloside IV and ferulic acid.

In certain embodiments, the agonist of receptor Na/K-ATPase/Src complex is found in one or more of: tan seng, red sage root, radix salvia miltiorrhizae, dang gui, angelica sinensis, huang qi and astragalus.

In certain embodiments, the compound is for the treatment of a disorder associated with one or more of cardiac hypertrophy, tissue fibrosis, congestive heart failure, cancer, wound or skin lesion.

In certain embodiments, the compound is identified for treatment of a disorder, the method further comprising: conducting an assay with the compound to: i) cause Src to be released from the receptor Na/K-ATPase complex if the compound is an agonist; or, ii) to prevent Src from the dissociation from the receptor Na/K-ATPase complex if the compound is an antagonist; and determining whether the compound is a Src activator or non ATP-competitive Src inhibitor, wherein the compound that significantly inhibits the disorder is a suitable compound for the treatment of the disorder.

In certain embodiments, the method includes diagnosing a subject with a disorder by: providing a sample from the subject; contacting the sample with a receptor NA/K-ATPase complex; and, determining the level of Src activity in the sample, as compared to a reference, wherein a difference in a level of the Src activity as compared to the reference, indicates that the subject has the disorder.

In certain embodiments, the method evaluating a treatment for a disorder, by: providing a sample from the subject; contacting the sample with the receptor NA/K-ATPase complex; administering one or more doses of a treatment; and, determining the level of Src activity in the sample, as compared to a reference, wherein a difference in a level of the Src activity, as compared to the reference indicates the efficacy of the treatment.

In certain embodiments, the method includes determining a subject's risk for development of a complication of a disorder, by: providing a sample from the subject; contacting the sample with a receptor NA/K-ATPase complex; and, determining the level of Src activity in the sample, as compared a reference, wherein a difference in a level of the Src, as compared to the reference, indicates the subject's risk of developing the complication.

In certain embodiments, the method includes of determining when a treatment modality administered to a subject to treat a disorder can be stopped, by: providing a sample from the subject; contacting the sample with a receptor NA/K-ATPase complex; and determining the level of the Src activity in the sample, as compared to a reference, wherein a level of the Src activity that approaches the level of the Src in the reference indicates whether the treatment can be stopped.

In certain embodiments, the method includes determining when a treatment for a disorder should be initiated in a subject, by: providing a sample from the subject; contacting the sample with a receptor NA/K-ATPase complex; and, comparing the level of Src activity in the sample, as compared to a reference, wherein a difference in a level of the Src activity, as compared to the reference, indicates whether the treatment should be initiated.

In certain embodiments, the sample is a cardiac cell, a cancer cell or a skin cell of the subject.

In certain embodiments, the reference represents a level of the Src activity prior to administration of the treatment.

In certain embodiments, the reference represents a level of Src activity in an unaffected subject.

In certain embodiments, the disorder is associated with one or more of cardiac hypertrophy, tissue fibrosis, congestive heart failure, cancer, wound or skin lesion.

In certain embodiments, the compound comprises a mixture of compounds.

In certain embodiments, the step of determining the level of Src activity in the sample comprises using one or more of: a high throughput assay; a FRET assay; a fluorescent polarization assay; a peptide-based FRET assay; or a cell based assay.

In certain embodiments, the cell based assay comprises a LLC-PK1 derived α1 knockdown PY-17 cell, a first control cell comprised of P11, and a second control cell comprised of AAC-19.

In certain embodiments, the first control cell P11 comprises LLC-PK1transfected with empty vector, and wherein the second control cell AAC-19 comprises rat α1-rescued PY-17 cells.

In certain embodiments, the cell based assay comprises a pair of cell lines, LL-A416P-4 and LL-A420P-20, wherein mutation of A420 to P results in inability of expressed Na/K-ATPase to bind and form a functional receptor complex as activated Src in A416P-rescued cells but not A420P mutant-rescued cells.

In certain embodiments, the cell based assay comprises cell lines (LY-I279A-3, LY-F286A-19), wherein expressed I279A or F286A mutant Na/K-ATPase is defective in conformational transition.

In certain embodiments, I279A and F286A mutants are defective in E1 to E2 and E2 to E1 conformational transition, respectively.

Also described herein are a FRET-based in vitro high throughput assay and a complementary cell culture based test. In one embodiment, the assay and/or test are useful for determining new agonists or antagonists of Na/K-ATPase/Src complex. Further, several new agonists and antagonists of this receptor complex using these newly developed assays are identified.

In another aspect, described herein are herbal medicinal preparations that comprise effective amounts one or more raw medicinal materials selected from: tan seng, red sage root, radix salvia miltiorrhizae, dang gui, angelica sinensis, huang qi and astragalus, wherein an agonist or antagonist of receptor Na/K-ATPase/Src complex found in each of the raw medicinal materials is present in an effective amount ranging from about 0.1 nM to about 10 nM.

In certain embodiments of the herbal medicinal preparation, the agonist comprises one or more of: ouabain, digoxin, marinobufagenin (MBG), oleic acid, docosahexaenoic (DHA), glutathione disulfide (GSSG) and allyl isothiocyanate

In certain embodiments of the herbal medicinal preparation, the antagonist comprises one or more of: 3,4,5-trihydroxyxanthone (MB5), 3,4,5,6-tetrahydroxyxanthone (MB7), curcumin, bisdemethoxycurcumin, tanshinone I, sodium danshensu, astragaloside IV, ferulic acid and tanshinone IIA.

In certain embodiments of the herbal medicinal preparation, the antagonist comprises one or more of: tanshinone I, sodium danshensu, astragaloside IV and ferulic acid.

In certain embodiments of the herbal medicinal preparation is for treatment of a disorder associated with one or more of cardiac hypertrophy, tissue fibrosis, congestive heart failure, cancer, wound or skin lesion.

In certain embodiments of the herbal medicinal composition is formulated for oral administration.

Various aspects of this invention will become apparent to those skilled in the art from the following detailed description of the preferred embodiment, when read in light of the accompanying drawings.

### BRIEF DESCRIPTION OF THE DRAWINGS

FIG. 1. Schematic illustration of Na/K-ATPase/Src receptor complex.

FIGS. 2A-2E. FRET based high throughput assay for agonist and antagonist:

FIG. 2A: Schematic illustration of agonist screening for Na/K-ATPase/Src receptor complex.

FIG. 2B: Schematic illustration of antagonist screening for Na/K-ATPase/Src receptor complex.

FIG. 2C: Graphs showing the antagonizing effects of MB5 effect on ouabain-induced Src activation in the re-constituted Na/K-ATPase/Src receptor complex system.

FIG. 2D: Graphs showing the antagonizing effects of tanshinone I and tanshinone IIA on ouabain-induced Src activation. The assays were done as in FIG. 2C.

FIG. 2E. Effects of oleic acid (OA) on Na/K-ATPase/Src complex.

FIGS. 3A-3B. Cell (LLC-PK1 and PY-17) based assay for agonist (ouabain and oleic acid):

FIG. 3A: Cells were treated with indicated concentration of ouabain for 5 min, and total cell lysates were analyzed for pY418 and total Src. A representative Western blot and combined quantitative data are shown.

FIG. 3B: Cells were treated with 20 μM OA for indicated time, then the total cell lysates were analyzed for pY418 and total Src. The graph shows the quantitative data. Values are mean±S.E. of at least three independent experiments. * p<0.05 compared with each control.

FIGS. 4A-4B. Cell (LL-A416P-4 and LL-A420P-20) based assay for agonist (ouabain). Cells were treated with indicated concentration of ouabain for 10 min, and total cell lysates were analyzed for pY418 and total Src:

FIG. 4A: Western blot.

FIG. 4B: Graph showing combined quantitative data. Values are mean±S.E. of at least three independent experiments. * p<0.05 compared with each control.

FIGS. 5A-5B. Cell (LY-I279A-3 and LY-F286A-19) based Assay for agonist (ouabain). Cells were treated with indicated concentration of ouabain for 10 min, and total cell lysates were analyzed for pY418 and total Src:

FIG. 5A: Western blot.

FIG. 5B: Graph showing combined quantitative data. Values are mean±S.E. of at least three independent experiments. * p<0.05 compared with each control.

FIGS. 6A-6B. MB5 as a Na/K-ATPase/Src receptor specific antagonist:

FIG. 6A: Effects of MB5 on ouabain induced activation of ERKs. A representative set of images is shown.

FIG. 6B. LLC-PK1 cells were pretreated with different concentrations of MB5 for 15 min, exposed to stimuli for either 10 or 3 min, and assayed for active ERKs. A representative set of images is shown.

FIGS. 6C-6E. Blocking ouabain-induced ERK activation by other antagonists of receptor Na/K-ATPase/Src complex.

FIG. 6C, Inhibition of ouabain-induced activation of ERKs by tashinone I. A representative set of images are presented from three separate experiments.

FIG. 6D. Western blot analyses showing the inhibitory effects of Ferulic Acid (A) on ouabain-induced ERK activation.

FIG. 6E. Western blot analyses showing the inhibitory effects of Tanshinone I (SI) or Astragaloside IV (B) on ouabain-induced ERK activation.

FIGS. 7A-7B. MB5 effect on Na/K-ATPase activity. The concentration curves of MB5 effect on the Na/K-ATPase activity (FIG. 7A (μM); FIG. 7B (nM)).

FIG. 7C. Effects of MB5 and tanshinone IIA on cancer cell growth. Left. MCF-7 cells were plated in 12-well cell culture plates at the density of 100,000 cells/well.

FIG. 7D. Effects of MB7 on prostate cancer cell growth. Cells were collected at each time point and the number was counted as in FIG. 7C.

FIGS. 8A-8C. MB5 effect on the growth of DU145 tumor xenografts in NOD/SCID mice Viable DU145 cells (5×10⁶) were injected to the flank of NOD/SCID mice:

FIG. 8A. Average body weight is shown after mice were killed.

FIG. 8B. Average tumor weight is shown after mice were killed.

FIG. 8C. Xenograft tumors taken from control and MB5-treated mice were photographed.

FIG. 9A. Non-limiting examples of agonists of the Na/K-ATPase/Src receptor.

FIG. 9B. Non-limiting examples of antagonists of the Na/K-ATPase/Src receptor.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Terms

It is to be understood that both the foregoing general description and the following detailed description are exemplary and explanatory only and are not intended to limit the scope of the current teachings. In this application, the use of the singular includes the plural unless specifically stated otherwise. In order to facilitate review of the various embodiments of the disclosure, the following explanations of specific terms are provided.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The use of the word “a” or “an” when used in conjunction with the term “comprising” in the claims and/or the specification may mean “one,” but it is also consistent with the meaning of “one or more,” “at least one,” and “one or more than one.”

Also, the use of “comprise”, “contain”, and “include”, or modifications of those root words, for example but not limited to, “comprises”, “contained”, and “including”, are not intended to be limiting. The term “and/or” means that the terms before and after can be taken together or separately. For illustration purposes, but not as a limitation, “X and/or Y” can mean “X” or “Y” or “X and Y”.

An “effective amount” or “therapeutically effective amount” is an amount sufficient to produce the desired effect, e.g., increased and/or decreased expression in comparison to the normal expression level detected in the absence of the specific composition. Increased/decreased expression a specific composition is achieved when the expression level is about 90%, 80%, 70%, 60%, 50%, 40%, 30%, 25%, 20%, 15%, 10%, 5%, or 0% greater/less than the relative expression level in a control sample. The desired effect of a specific composition may also be measured by detecting an increase in the expression targeted by the specific composition.

By “modulate” is meant that the expression, or level, or activity is up-regulated or down-regulated, such that expression, level, or activity is greater than or less than that observed in the absence of the modulator. For example, the term “modulate” can mean “inhibit,” but the use of the word “modulate” is not limited to this definition.

The term “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB.

The terms “agent” and “drug” generally refer to any therapeutic agents (e.g., chemotherapeutic compounds and/or molecular therapeutic compounds), antisense therapies, radiation therapies, or surgical interventions, used in the treatment of a particular disease or disorder.

The term “adjunctive therapy” generally refers to a treatment used in combination with a primary treatment to improve the effects of the primary treatment.

The term “clinical outcome” generally refers to the health status of a subject following treatment for a disease or disorder, or in the absence of treatment. Clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy.

The term “decrease in survival” generally refers to a decrease in the length of time before death of a subject, or an increase in the risk of death for the subject.

The term “control” generally refers to a sample or standard used for comparison with an experimental sample, such as a sample obtained from a subject. In some embodiments, the control is a sample obtained from a healthy subject. In some embodiments, the control is cell/tissue sample obtained from the same subject. In some embodiments, the control is a historical control or standard value (i.e., a previously tested control sample or group of samples that represent baseline or normal values, such as the level in a control sample). In other embodiments, the control is a sample obtained from a healthy subject, such as a donor. Test samples and control samples can be obtained according to any method known in the art.

The terms “prevent,” “preventing” and “prevention” generally refer to a decrease in the occurrence of disease or disorder in a subject. The prevention may be complete, e.g., the total absence of the disease or disorder in the subject. The prevention may also be partial, such that the occurrence of the disease or disorder in the subject is less than that which would have occurred without embodiments of the present invention. “Preventing” a disease generally refers to inhibiting the full development of a disease.

The terms “treating” and/or “ameliorating a disease” generally refer to a therapeutic intervention that ameliorates a sign or symptom of a disease or disorder after it has begun to develop. “Ameliorating” generally refers to the reduction in the number or severity of signs or symptoms of a disease or disorder.

The term “subject” includes human and non-human animals. The preferred subject for treatment is a human. “Subject” and “subject” are used interchangeably herein.

The term “therapeutic” generally is a generic term that includes both diagnosis and treatment.

The term “therapeutic agent” generally refers to a chemical compound, small molecule, or other composition, capable of inducing a desired therapeutic or prophylactic effect when properly administered to a subject. Multiple therapeutic agents may be used simultaneously or in sequence.

As used herein, a “candidate agent” is a compound selected for screening to determine if it can function as a therapeutic agent. “Incubating” includes a sufficient amount of time for an agent to interact with a cell or tissue. “Contacting” includes incubating an agent in solid or in liquid form with a cell or tissue. “Treating” a cell or tissue with an agent includes contacting or incubating the agent with the cell or tissue.

The term “therapeutically effective amount” generally refers to that amount of the therapeutic agent sufficient to result in amelioration of one or more symptoms of a disorder, or prevent advancement of a disorder, or cause regression of the disease or disorder. A “therapeutically effective amount” can be a quantity of a specified pharmaceutical or therapeutic agent sufficient to achieve a desired effect in a subject, or in a cell, being treated with the agent. For example, this can be the amount of a therapeutic agent that alters the expression of miR/s, and thereby prevents, treats or ameliorates the disease or disorder in a subject. The effective amount of the agent will be dependent on several factors, including, but not limited to the subject or cells being treated, and the manner of administration of the therapeutic composition.

As used herein, “pharmaceutical compositions” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is incorporated herein by reference.

The term “pharmaceutically acceptable vehicles” generally refers to such pharmaceutically acceptable carriers (vehicles) as would be generally used. Remington's Pharmaceutical Sciences, by E. W. Martin, Mack Publishing Co., Easton, Pa., 20 Edition, describes compositions and formulations suitable for pharmaceutical delivery of one or more therapeutic compounds, molecules or agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The term “pharmaceutically acceptable salt” generally refers to any salt (e.g., obtained by reaction with an acid or a base) of a compound of embodiments of the present invention that is physiologically tolerated in the target animal (e.g., a mammal). Salts of the compounds of embodiments of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of embodiments of the invention and their pharmaceutically acceptable acid addition salts. Examples of bases include, but are not limited to, alkali metal (e.g., sodium) hydroxides, alkaline earth metal (e.g., magnesium) hydroxides, ammonia, and the like. Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of embodiments of the present invention compounded with a suitable cation such as Na+, NH4+, and NW4+(wherein W is a C1-4 alkyl group), and the like. For therapeutic use, salts of the compounds of embodiments of the present invention are contemplated as being pharmaceutically acceptable. However, salts of acids and bases that are non-pharmaceutically acceptable may also find use, for example, in the preparation or purification of a pharmaceutically acceptable compound.

As used herein, the terms “compound” and “agent” are generally deemed to be synonymous and thus interchangeable unless suggested otherwise by context.

As used herein, the term “substantially pure” means that the material is substantially free of the sequences and/or molecules with which it is associated in its natural state, and those molecules used in the isolation procedure. The term “substantially pure” also encompasses purification of a polynucleotide or a polypeptide to near homogeneity. The term “substantially free” means that the sample is at least 50%, preferably at least 70%, more preferably 80%, even more preferably 90%, and most preferably 99% free of the materials and compounds with which is it associated in nature.

Broad Description

This invention is based, at least in part, on the inventors' discovery that the Na/K-ATPase binds and inhibits Src. As used herein, “Src” refers to a non-receptor tyrosine kinase.

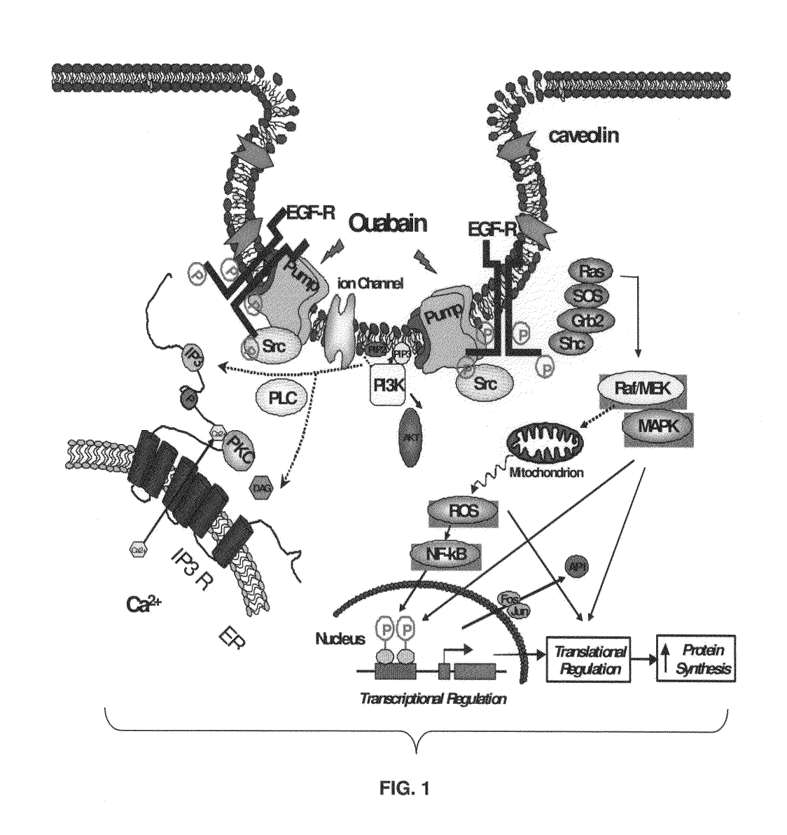

Referring first to FIG. 1, there is shown an illustration of Na/K-ATPase/Src receptor complex. The definitions in FIG. 1 include: EGFR, epidermal growth factor receptor; IP3R, IP3 receptor; PI3K, phosphatidylinositol 3-kinase; PKC protein kinase C; PLC phospholipase C; Grb2, growth factor receptor-bound protein 2; SOS, son of sevenless; Shc, Src homology collagen-like protein; MAPK, mitogen-activated protein kinase; MEK, MAPK-ERK activating kinase; ROS, reactive oxygen species.

As illustrated in FIG. 1, the receptor complex resides in caveolae. Binding of ouabain to the receptor complex leads to the activation of Src, then transactivation of EGFR and subsequent stimulation of several proteins and lipid cascades. In non-limiting examples, the receptor agonists are useful for wound healing and anti-wrinkle uses. In non non-limiting examples, the receptor antagonists are useful for cancer, fibrosis, CHF and CRF.

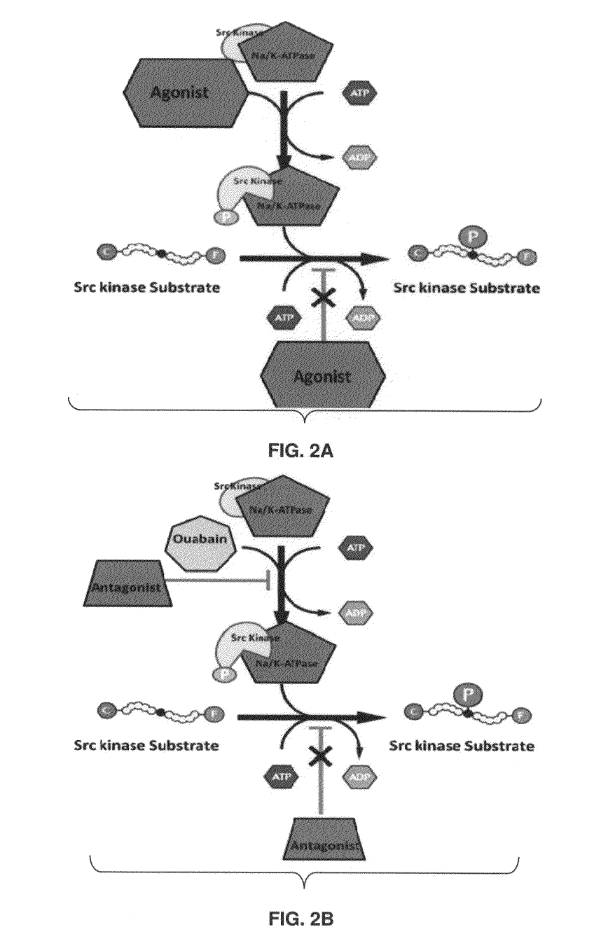

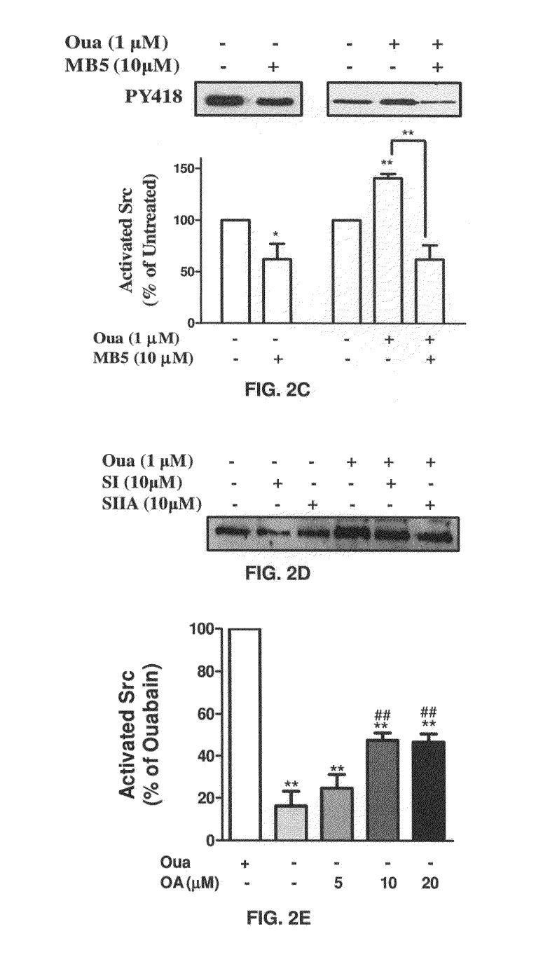

Referring now to FIGS. 2A and 2B, there are shown schematic illustrations of a FRET based high throughput assay for agonist and antagonist. FIG. 2A shows a schematic illustration of agonist screening for Na/K-ATPase/Src receptor complex. A pathway-based approach is useful to screen for agonists of Na/K-ATPase/Src receptor targets. Agonist can interact with Na/K-ATPase resulting Src phosphorylation. This active Src transfers the γ-phosphate of ATP to a single tyrosine residue in the synthetic peptide substrate. Then, a site specific protease recognizes and cleaves non-phosphorylated peptides. Phosphorylated peptides exhibit suppressed cleavage by the Development Reagent. Cleavage disrupts FRET on the peptide, whereas uncleaved, phosphorylated peptides maintain FRET.

FIG. 2B shows a schematic illustration of antagonist screening for Na/K-ATPase/Src receptor complex. This assay is similar as the agonist screening. For example, in the presence of ouabain, antagonist can reduce ouabain induced peptide substrate phosphorylation.

In one example, the FRET-based in vitro high throughput assay utilizes the in vitro reconstitution of receptor Na/K-ATPase and Src, and a peptide Src substrate-based FRET assay. The α1 Na/K-ATPase was purified from pig kidney and the preparations that exhibit specific activity higher than 800 μmol Pi/mg protein/h were used in the assay. To reconstitute the receptor α1 Na/K-ATPase/Src complex, purified pig kidney Na/K-ATPase was mixed with purified recombinant Src. This receptor complex has an inhibited Src activity. The binding of agonists to the receptor complex releases the inhibited Src and result in an increase in Src activity, which is measured by a peptide-based FRET analysis (FIG. 2A, FIG. 2B). To screen the agonist of receptor Na/K-ATPase/Src complex, the reconstituted receptor is exposed to compounds, and then measured for Src activation using the peptide-based FRET assay.

For Example, FIG. 2C shows the antagonizing effects of MB5 effect on ouabain-induced Src activation in the re-constituted Na/K-ATPase/Src receptor complex system for screening receptor agonist or antagonist. Phosphorylation of Src pY 418, as an indicator of Src activation, was measured by Western blot. Ouabain was used at 1 μM and the assay was done in the presence of 100 μM vanade to inactivate ATPase activity. Values are mean±S.E. of at least three independent experiments. * p<0.05 compared with control. ** p<0.01 compared with control.

FIG. 2D shows the antagonizing effects of tanshinone I and tanshinone IIA on ouabain-induced Src activation. The assays were done as in FIG. 2C.

The effects of oleic acid (OA) on Na/K-ATPase/Src complex are shown in FIG. 2D and FIG. 2E. Different concentration of OA was incubated with Na/K-ATPase/Src receptor for 15 min before adding ATP and then assay for pY418 phosphorylation. Values are mean±S.E. of at least three independent experiments. ** p<0.01 compared with ouabain group. ## p<0.01 compared with no-treated group.

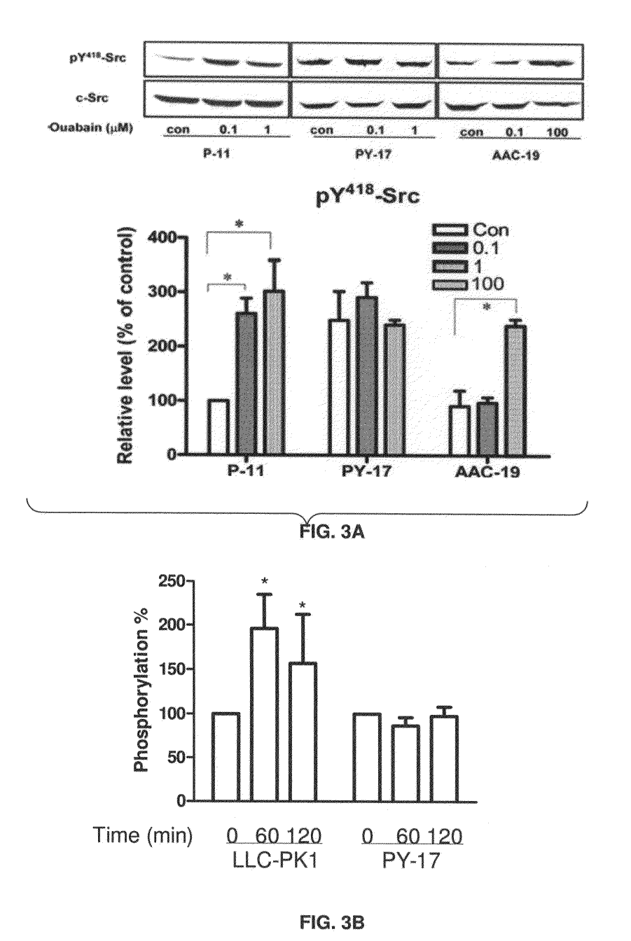

To confirm that these agonists activate Src through the Na/K-ATPase, three complementary cell-based assays were established. One assay uses a LLC-PK1 derived al knockdown PY-17 cell, a control cell P11 (LLC-PK1transfected with empty vector), and another control cell AAC-19 (rat al-rescued PY-17 cells). While these compounds activate Src in P11 and AAC-19 cells, knockdown of Na/K-ATPase abolished their effects in PY-17 cells, confirming that the Na/K-ATPase is indeed the receptor as demonstrated by ouabain exposure (FIG. 3A).

Similarly, oleic acid activated Src in LLC-PK1 cells but not PY-17 cells (FIG. 3B). The second assay uses another pair of cell lines, LL-A416P-4 and LL-A420P-20. These cells were rat α1 A416P and A420P mutant-rescued PY-17 cells.

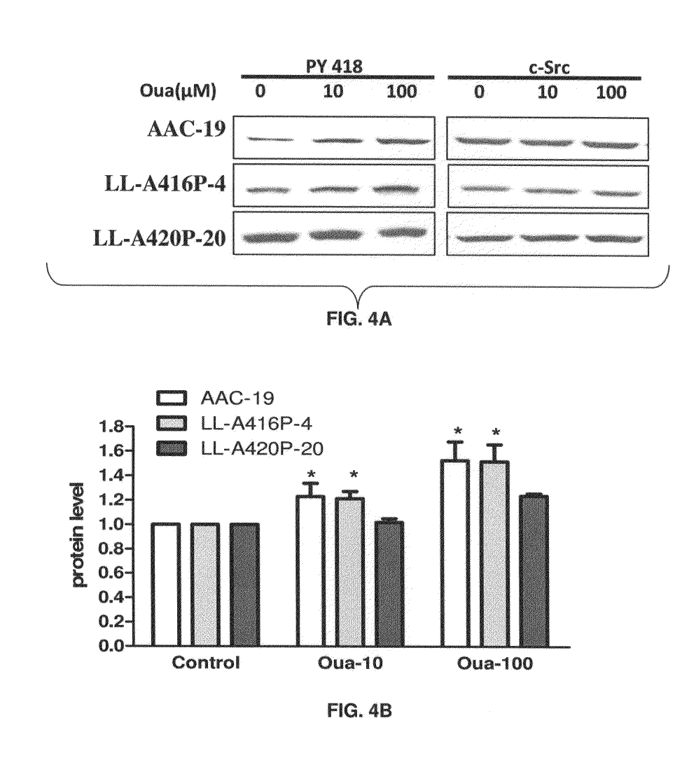

As shown in FIGS. 4A-4B, mutation of A420 to P resulted in inability of expressed Na/K-ATPase to bind and form a functional receptor complex as ouabain activated Src in A416P-rescued cells but not A420P mutant-rescued cells. Cell (LL-A416P-4 and LL-A420P-20) based assay for agonist (ouabain): Cells were treated with indicated concentration of ouabain for 10 min, and total cell lysates were analyzed for pY418 and total Src. FIG. 4A shows a Western blot, and FIG. 4B shows the combined quantitative data. Values are mean±S.E. of at least three independent experiments. * p<0.05 compared with each control.

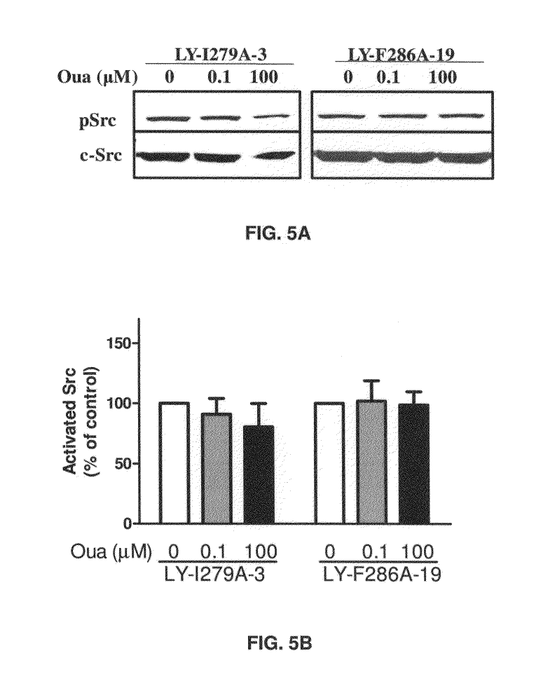

The third assay uses other two cell lines (LY-I279A-3, LY-F286A-19), where the expressed I279A or F286A mutant Na/K-ATPase was defective in conformational transition (I279A and F286A mutants were defective in E1 to E2 and E2 to E1 conformational transition, respectively). As shown in FIG. 5, once the conformational transition is inhibited, ouabain could no longer activate Src in these mutant cells. Cell (LY-I279A-3 and LY-F286A-19) based Assay for agonist (ouabain): Cells were treated with indicated concentration of ouabain for 10 min, and total cell lysates were analyzed for pY418 and total Src. FIG. 5A shows a Western blot, and FIG. 5B shows the combined quantitative data. Values are mean±S.E. of at least three independent experiments. * p<0.05 compared with each control.

To assay for receptor antagonist, the reconstituted receptor was exposed to compounds in the presence and absence of an agonist (ouabain) and then assessed whether the compounds could antagonize ouabain-induced activation of Src. To verify that they are specific antagonists of receptor Na/K-ATPase/Src complex, their effects on ouabain-, EGF- and dopamine-induced signal transduction was measured in LLC-PK1 and PY-17 cells.

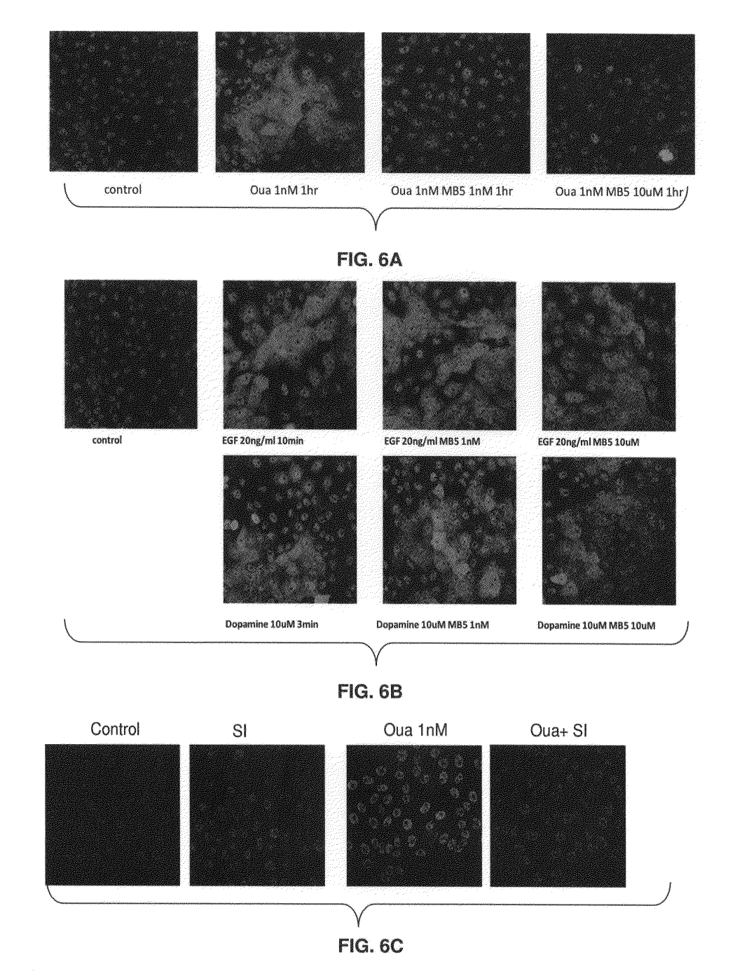

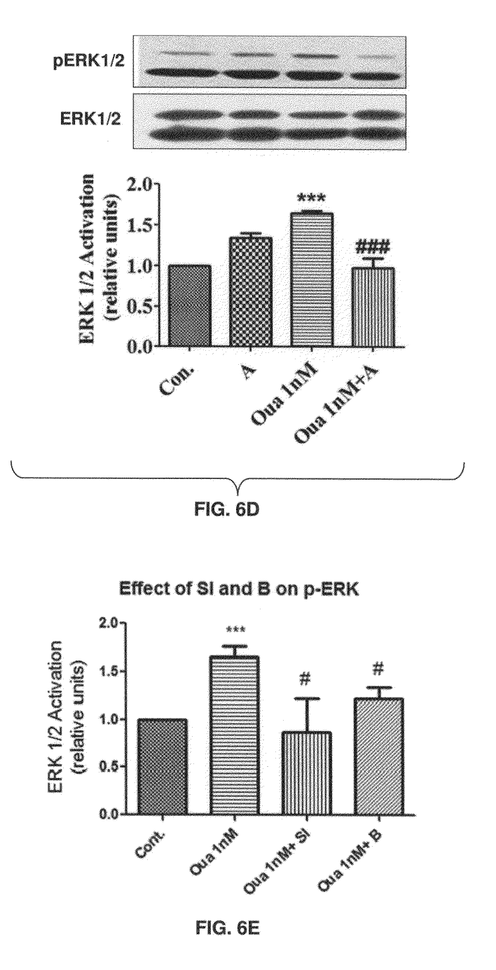

The data presented in FIGS. 6A-6E show that MB5 blocked ouabain, but not EGF- and dopamine-induced activation of ERKs.

MB5 as a Na/K-ATPase/Src receptor specific antagonist is shown in FIGS. 6A-6B. FIG. 6A shows the effects of MB5 on ouabain induced activation of ERKs. LLC-PK1 cells were pretreated with different concentrations of MB5 for 15 min, exposed to 1 nM ouabain for 60 min, and assayed for active ERKs. A representative set of images is shown. As shown in FIG. 6B. LLC-PK1 cells were pretreated with different concentrations of MB5 for 15 min, exposed to stimuli for either 10 or 3 min, and assayed for active ERKs. A representative set of images is shown.

The data presented in FIGS. 6C-6E show blocking ouabain-induced ERK activation by other antagonists of receptor Na/K-ATPase/Src complex. FIG. 6C shows the inhibition of ouabain-induced activation of ERKs by tashinone I. LLC-PK1 cells were pre-treated with Tanshinone I for 30 minutes. Both control and Tanshinone I-pretreated cells were exposed to ouabain (1 nM) for 1 hour, and processed for immuno-staining of active ERK as in FIG. 6A. A representative set of images are presented from three separate experiments.

FIG. 6D shows Western blot analyses showing the inhibitory effects of Ferulic Acid (A) on ouabain-induced ERK activation. LLC-PK1 cells were serum-starved overnight, washed and pretreated with Ferulic acid (A) for 30 min. Both control and compounds-pretreated cells were exposed to ouabain (1 nM) for 1 h. Cell lysates were collected, separated by SDS-PAGE and probed for activated-ERK and total-ERK. A representative western blot is shown. The quantitative data from three to four separate experiments were presented. (***, p<0.001 vs control; #, p<0.05 vs Oua).

FIG. 6E shows Western blot analyses showing the inhibitory effects of Tanshinone I (SI) or Astragaloside IV (B) on ouabain-induced ERK activation. The experiments were done as in FIG. 6D. The quantitative data from four separate experiments were presented. (***, p<0.001 vs control; ## #, p<0.001 vs Oua).

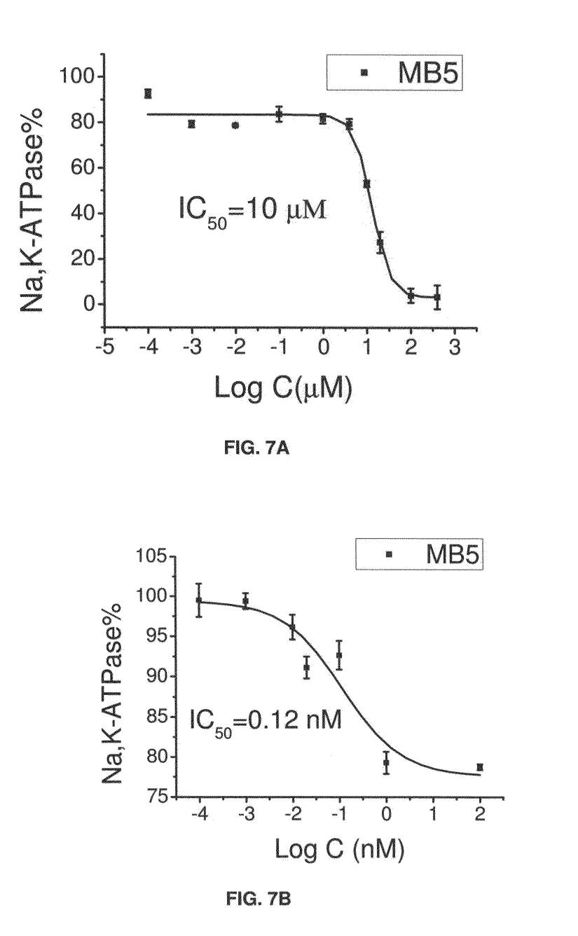

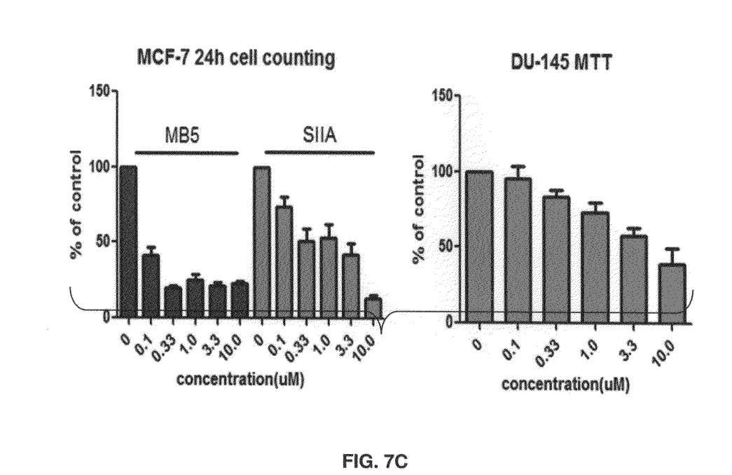

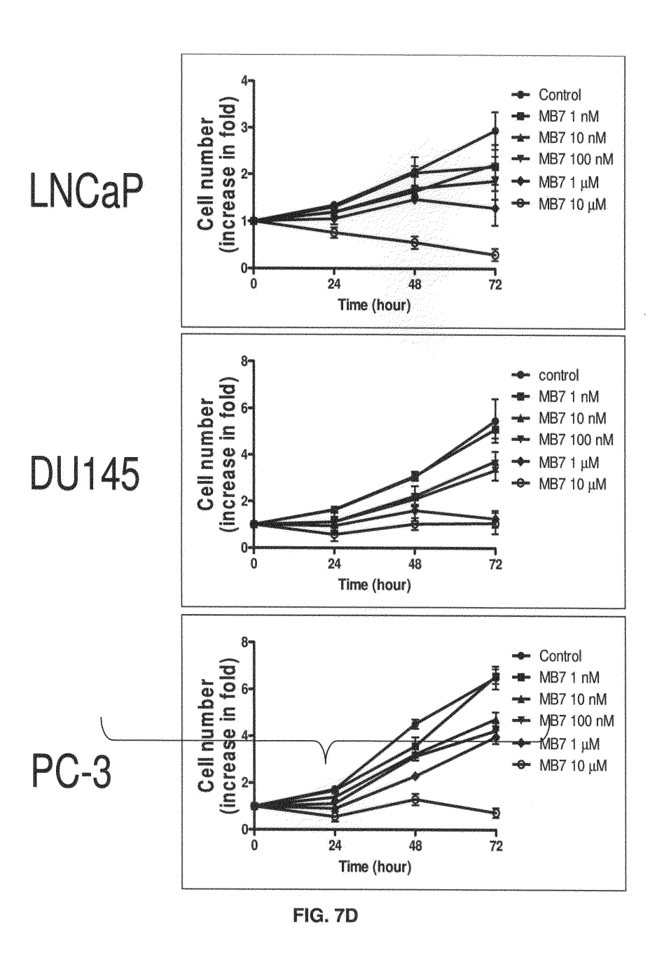

Referring now to FIGS. 7A-7D, purified pig Na/K-ATPase was used to test whether they affect the enzymatic activity.

The MB5 effect on Na/K-ATPase activity is shown in FIGS. 7A-7B. The concentration curves of MB5 effect on the Na/K-ATPase activity (FIG. 7A (μM); FIG. 7B (nM)). The purified Na/K-ATPase was incubated with different concentration of MB5 for 15 min, then assayed for ATPase activity. The data are combined from three to five separate experiments and are presented as means±S.E.

FIG. 7C shows the effects of MB5 and tanshinone IIA on cancer cell growth. Left. MCF-7 cells were plated in 12-well cell culture plates at the density of 100,000 cells/well. Cells were treated with MB5 or Tanshinone IIA (SIIA) as indicated for 24 h, collected and counted. Right. DU-145 cells were cultured in 96-well plates at 3000 cells/well overnight. Cells were treated with Tanshinone IIA at the indicated concentrations for 72 h and analyzed by MTT assay.

FIG. 7D shows the effects of MB7 on prostate cancer cell growth. Cells were plated in 12-well plates and treated with different concentrations of MB7 as indicated. Cells were collected at each time point and the number was counted as in FIG. 7C.

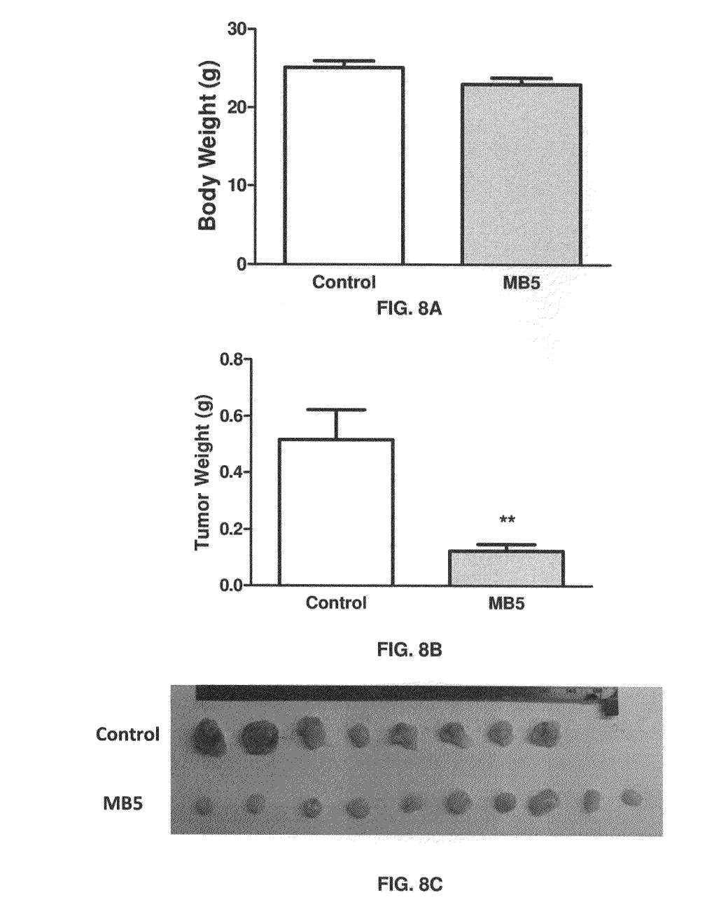

FIGS. 8A-8C show the MB5 effect on the growth of DU145 tumor xenografts in NOD/SCID mice Viable DU145 cells (5×10⁶) were injected to the flank of NOD/SCID mice. After tumor reached 100 mm³, mice were injected via i.p. with DMSO or MB5 at a dose of 20 mg/Kg. Values are mean±S.E. ** p<0.01 compared with each control. FIG. 8A shows the average body weight is shown after mice were killed. FIG. 8B shows the average tumor weight is shown after mice were killed. FIG. 8C shows xenograft tumors taken from control and MB5-treated mice were photographed.

It is to be noted that MB5 and MB7 are derivatives of xanthone and antagonize ouabain-induced signal transduction in cultured cells. Moreover, when MB5 was administered, it inhibited the growth of Du145 tumor xenograft in mice (FIG. 8).

The second class is curcumin and its derivative bisdemethoxycurcumin. Tanshinone I, danshensu, astragaloside IV, ferulic acid and tanshinone IIA were also identified. These compounds are isolated from the traditional Chinese medicines.

While the antagonists are useful to inhibit tumor cell growth, cardiac hypertrophy and organ fibrosis, the identified agonists are also useful to protect organs from ischemia/reperfusion injury, to accelerate wound healing and to prevent aging-related skin wrinkle

The assays are cost effective. An in vitro high throughput assay is used to identify the target, which is then followed by complementary cell-based analyses that verify the molecular target in living cells being indeed the Na/K-ATPase.

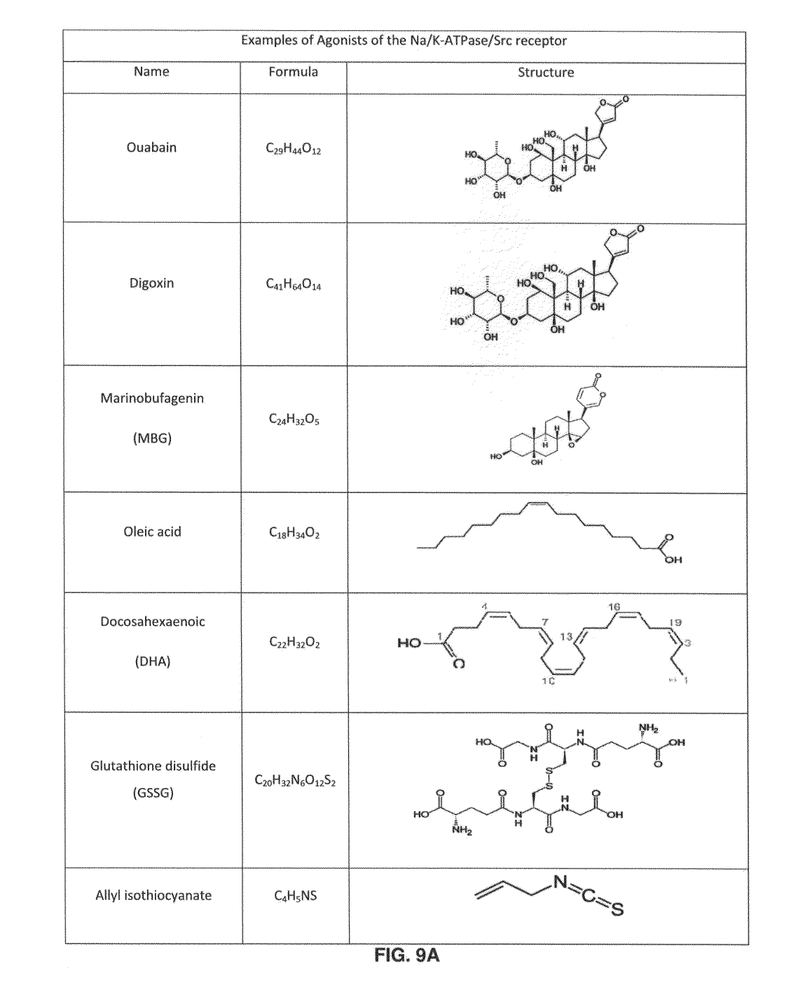

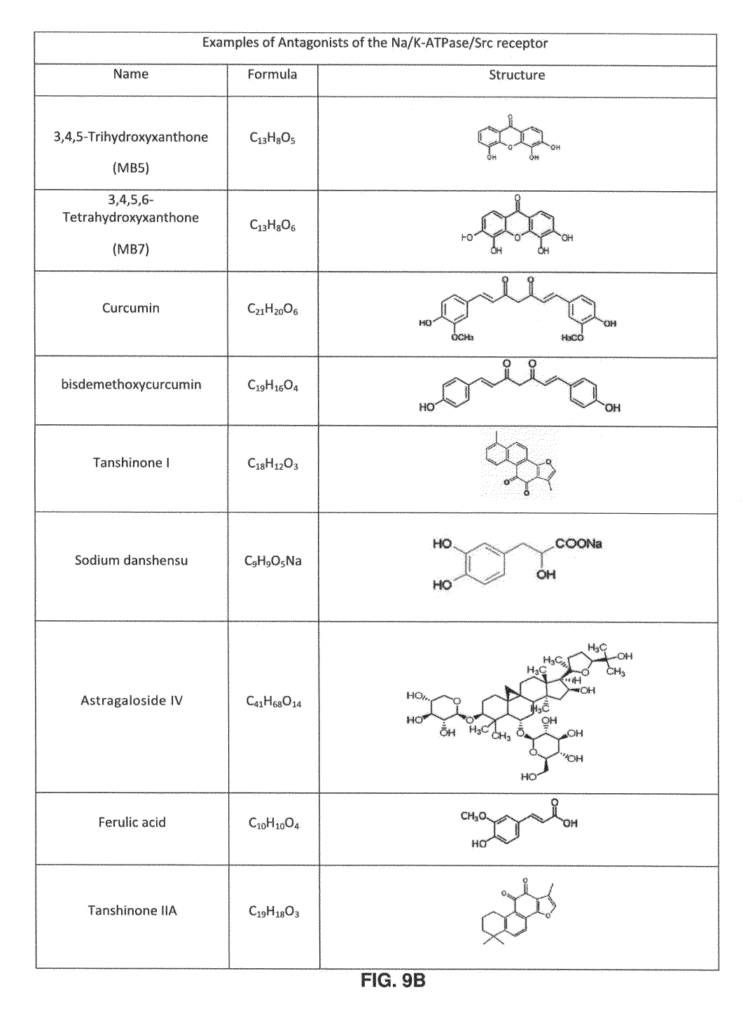

It also is to be noted that, in addition to MB5, the compounds shown in FIG. 9B are positively identified as ouabain antagonists using these assays. That is, FIG. 9A and FIG. 9B provide non-limiting examples of the names and structures of various active compounds. To complement, the positively identified compounds were subjected to an in vitro kinase assay where the activation of Src was measured by pY418 antibody.

### Examples

The present invention is further defined in the following Examples, in which all parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. All publications, including patents and non-patent literature, referred to in this specification are expressly incorporated by reference. The following examples are intended to illustrate certain preferred embodiments of the invention and should not be interpreted to limit the scope of the invention as defined in the claims, unless so specified.

Materials:

Z′-LYTETM Kinase assay kit-tyr 2 peptide was purchased from Invitrogen (Camarillo, Calif.). ATP and ouabain were obtained from Sigma (St. Louis, Mo.). Biomol Green was purchased from BIOMOL (Plymouth Meeting, Pa.). Purified recombinant Src was obtained from Upstate Biotechnology (Lake Placid, N.Y.). Polyclonal anti-Tyr (P) 418-Src was obtained from Invitrogen (Camarillo, Calif.). Anti-c-Src (B-12) monoclonal antibody was from Santa Cruz Biotechnology Inc. (Santa Cruz, Calif.). The chemicals were of the highest purity available. Fresh pig kidneys were purchased from a nearby slaughterhouse, and stored at −80° C. until used for enzyme preparation.

High Throughput Screen Assay:

The chemical library used for screening in the present study contained 100 structurally diverse, drug-like, naturally occurring organic compounds or their semisynthetic derivatives. Stock compounds were prepared at 10 mM in DMSO. The specific activities of Na/K-ATPase of various kidney preparations were in the range of 900-1,200 μmol/mg/h. The high throughput screen was conducted in a Corning 384-well low-volume assay plates at room temperature.

The first step was Kinase Reaction with the final reaction volume of 10 μl. The purified Src (4.5 U) was incubated with 2 μg of the purified Na/K-ATPase in phosphate-buffered saline (PBS) for 5 min. Afterward, the Na/K-ATPase/Src complex was exposed to compounds to screen for agonist or ouabain 10 μM plus compounds to screen antagonists for 10 min. Then, the Src kinase substrate Tyr 2 peptide or phosphor-peptide (control) was added. The reaction was initiated by addition of 2 mM ATP/Mg²⁺, continued for 60 min.

The second step was Development Reaction. Each well was added 5 μl Development Solution, then mix the plate and incubate another 60 min. The assay was stopped by adding 5 μl stop reagent and then fluorescent signals were measured.

Cell Culture:

The pig kidney epithelia cells (LLC-PK1 cells) were obtained from ATCC and maintained in Dulbecco's modified Eagle's medium (DMEM) in the presence of 10% FBS, 100 units/ml penicillin, and 100 μg/ml streptomycin in a 5% CO2 humidified incubator. To eliminate the confounding effect of growth factors in the serum, cells were serum starved over night before experiments.

Referring now to FIG. 3A, cells were treated with indicated concentration of ouabain for 5 min, and total cell lysates were analyzed for pY418 and total Src. FIG. 3B shows where cells were treated with 20 μM OA for indicated time, then the total cell lysates were analyzed for pY418 and total Src. The graph shows the quantitative data. Values are mean±S.E. of at least three independent experiments. * p<0.05 compared with each control.

Western Blot Analysis:

Cells were washed with PBS and solubilized in modified ice-cold radioimmune precipitation assay buffer, and subjected to Western blot analysis. Protein signals were detected using an ECL kit and quantified using ImageJ.

Kinase Activity Assay of Src:

To determine whether the compound affect the Na/K-ATPase/Src receptor activity, the compounds was incubated with the purified Src (4.5 U) and 1 μg Na/K-ATPase for 15 min at 37° C. Afterward, 2 mM ATP/Mg²⁺ was added. The reaction continued for 15 min at 37° C. and was stopped by addition of SDS sample buffer. Afterward, the Src pY418 was measured by anti-pY418 antibody to indicate Src activation.

ATPase Activity:

Na/k-ATPase activity was measured. Briefly, purified pig kidney Na/K-ATPase (specific activities between 900-1,200 μmol Pi/mg/h) was incubated in the buffer containing 20 mM Tris (pH 7.4), 1 mM EGTA, 3 mM MgCl2, 12.5 mM KCl, 100 mM NaCl. After compounds were added, mixtures were incubated for 15 min at 37° C. and reaction was initiated by adding 2 mM ATP·Mg. Reactions were carried out for 10 min and then stopped by the addition of 100 μl ice-cold trichloroacetic acid. Reaction mixtures were assayed for released phosphate using the BIOMOL GREEN™ Reagent according to the manufacturer's instructions. Na/K-ATPase activities were calculated as the difference between the presence and absence of 1 mM ouabain.

Immunostaining Assay for Active ERK:

LLC-PK1 cells grown on coverslip were serum-starved and treated with MB5 or stimuli for indicated time. Immunostaining of phosphor-ERK was performed with commercial ERK/MAPK (phospho-Thr202/Tyr204) phosphorylation/translocation cell-based assay kit (Cayman Chemical) according to the manufacturer's instructions. The signals were detected by a Leica (Wetzlar, Germany) confocal microscope. Leica confocal software was used for data analysis.

DU145 Xenograft Tumors in NOD/SCID Mice:

Animal protocols were approved by the Institutional Animal Care and Use Committee at the University of Toledo Health Science Campus. Tumor xenografts were established by subcutaneous injection of 5×10⁶ DU145 cells into two flanks of 6-week-old female NOD/SCID mice (Charles River). When the tumors reached an average volume of 100 mm³, mice were injected intraperitoneally with DMSO or MB5 (at doses of 20 mg/kg body weight) every day for 3 weeks.

Data Analysis:

Data are given as mean±S.E. Statistical analysis was performed using the Student's t test and significance was accepted at p<0.05.

Herbal Preparations and Compositions

As used herein, an “herb” refers to any plant that is reputed to have medicinal value in Traditional Chinese Medicine (TCM). For example, the use of extracts of various parts of these plants have been passed down from ancient to modern Chinese practitioners of herbal medicine as a means for treating various ailments. While each of the herbs, and parts thereof, that make up the pharmaceutical compositions of this invention have long been known in TCM, use of an extract or combination of extracts in a composition as disclosed herein for the treatments described has not been previously disclosed.

Without wishing to be bound by any particular theory, the herbal compositions and preparations thereof are particularly useful for treating or lessening the severity of a disease, condition, or disorder where alteration of Src activity is implicated in the disease, condition, or disorder.

Without wishing to be bound by any particular theory, it is now proposed herein that the herbal preparations and pharmaceutically acceptable compositions thereof may alter Src activity.

Accordingly, another embodiment of the present invention relates to a method of altering Src activity in a patient in need thereof, where the method comprises administering to the patient an herbal preparation or pharmaceutically acceptable compositions thereof.

Yet another embodiment relates to a method of suppressing the activation of Src in cells in a patient in need thereof, wherein the method comprises administering to the patient an herbal preparation or pharmaceutically acceptable composition thereof.

In certain embodiments, the herbal preparation comprises a mixture of plants and/or plant extracts. Such herbal preparation provides an additive and synergistic effect where the nature of the different agonists or antagonists in each plant and/or plant extracts provides a beneficial effect, yet does not adversely affect the patient by causing an overdosage of other ingredients in such plants and/or plant extracts.

By evaluating each herbal preparation for effective amounts of agonists/antagonist of the receptor Na/K-ATPase/Scr complex, there is now achieved a therapeutic, as well as balanced, treatment. In particular, the present method described herein provides an accurate method for determining the amount or agonist/antagonist in a particular plant, plant extract or herbal preparation. Thus, an herbal medicinal preparation can that comprise effective amounts one or more raw medicinal materials that have an agonist or antagonist of receptor Na/K-ATPase/Src complex is present in a desired effective amount.

In certain non-limiting examples, the effective amount ranges from about 0.1 to about 10 nM. In other embodiments, the effective amount is administered in an amount not greater 10 nM. In other embodiments, the effective amount is administered in an amount not greater 1 nM. In other embodiments, the effective amount is administered for at least about 5 hours. In other embodiments, the effective amount is administered in an amount not greater 1 nM. In other embodiments, the effective amount is administered for at least about 5 hours.

The herbal preparations and pharmaceutically acceptable compositions thereof may be employed to treat existing symptoms (i.e., to reduce the severity, intensity, and/or duration of such symptoms). In such cases, the formulas or compositions thereof are administered to an individual after the symptoms have developed.

Alternatively or additionally, the herbal preparations may be used to prevent or delay the onset of symptoms in an individual who has previously suffered, or to reduce the severity, intensity, or duration of subsequently-developed symptoms.

The herbal preparations and compositions thereof may also be administered prior to the development of a disorder.

In other embodiments of the present invention, the herbal preparation can be administered in combination with one or more additional therapeutic agents. For example, the herbal preparations may be administered in combination with one or more cardiotonic steroids.

In certain embodiments, the herbal preparations are administered within a pharmaceutically acceptable composition, thus forming a single dosage form. In other embodiments, the herbal preparations are administered contemporaneously with one or more additional pharmaceutically acceptable compositions as a separate dosage form.

It is also to be appreciated that the herbal preparations and pharmaceutically acceptable compositions can be employed in combination therapies, that is, the herbal preparations and pharmaceutically acceptable compositions thereof can be administered concurrently with, prior to, or subsequent to, one or more other desired therapeutics or medical procedures.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the herbal preparations may be administered concurrently with another agent used to treat the same disorder), or they may achieve different effects (e.g., control of any adverse effects).

As used herein, additional therapeutic agents that are normally administered to treat, lessen the severity of, or prevent a particular disease, or condition, are known as “appropriate for the disease, or condition, being treated.”

The herbal preparations may be administered to a subject in combination with one or more other therapeutic treatments. For example, cardiotonic steroid administration is an established and accepted treatment for cardiac hypertrophy, tissue fibrosis, congestive heart failure, cancers and wound or skin lesions. Thus, the herbal preparations may desirably be administered in combination with standard or reduced treatments, whether oral or systemic.

In another aspect, described herein are herbal medicinal preparations that comprise effective amounts one or more raw medicinal materials selected from: tan seng, red sage root, radix salvia miltiorrhizae, dang gui, angelica sinensis, huang qi and astragalus, wherein an agonist or antagonist of receptor Na/K-ATPase/Src complex found in each of the raw medicinal materials is present in an effective amount ranging from about 0.1 nM to about 10 nM.

In certain embodiments of the herbal medicinal preparation, the agonist comprises one or more of: ouabain, digoxin, marinobufagenin (MBG), oleic acid, docosahexaenoic (DHA), glutathione disulfide (GSSG) and allyl isothiocyanate

In certain embodiments of the herbal medicinal preparation, the antagonist comprises one or more of: 3,4,5-trihydroxyxanthone (MB5), 3,4,5,6-tetrahydroxyxanthone (MB7), curcumin, bisdemethoxycurcumin, tanshinone I, sodium danshensu, astragaloside IV, ferulic acid and tanshinone IIA.

In certain embodiments of the herbal medicinal preparation, the antagonist comprises one or more of: tanshinone I, sodium danshensu, astragaloside IV and ferulic acid.

In certain embodiments of the herbal medicinal preparation is for treatment of a disorder associated with one or more of cardiac hypertrophy, tissue fibrosis, congestive heart failure, cancer, wound or skin lesion.

Modes of Administrations

In certain embodiments of the herbal medicinal composition is formulated for oral administration.

An herbal preparation can be administered to a patient either as a “tea,” without combination with any other substances or further manipulation, or it can be administered as a pharmaceutical composition where the extract is mixed with suitable carriers or recipient(s). In treating a patient exhibiting a disorder of interest, a therapeutically effective amount of the extract is administered. A therapeutically effective amount refers to that amount of the extract that results in amelioration of symptoms or a prolongation of survival in a patient.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

The publication and other material used herein to illuminate the invention or provide additional details respecting the practice of the invention, are incorporated by reference herein, and for convenience are provided in the following bibliography.

Citation of the any of the documents recited herein is not intended as an admission that any of the foregoing is pertinent prior art. All statements as to the date or representation as to the contents of these documents is based on the information available to the applicant and does not constitute any admission as to the correctness of the dates or contents of these documents.

1. A method of identifying a compound that alters Src activity, the method comprising:
i) purifying α1 Na/K-ATPase to obtain Na/K-ATPase exhibiting specific activity higher than 800 μmol Pi/mg protein/h;
ii) mixing the purified Na/K-ATPase of step (i) with Src to form a receptor Na/K-ATPase/Src complex having inhibited Src activity;
iii) exposing the receptor Na/K-ATPase/Src complex of step (ii) to a compound, wherein binding of the compound releases the inhibited Src from the receptor Na/K-ATPase/Src complex, resulting in an increase in Src activity; and
iv) measuring the increase in Src activity, wherein the increased Src activity is indicative of the compound altering Src activity.

2. The method of claim 1, wherein the step of measuring comprises determining whether there is an enhanced level of Src activity as compared to a control level of Src activity,
wherein an increase in the level of Src activity indicates that the compound is an agonist, and,
wherein a decrease in the level of Src activity relative to the control level indicates that the compound is an antagonist.

3. The method of claim 2, wherein the agonist compound of receptor Na/K-ATPase/Src complex comprises at least one of:

| Agonist Compound Name | Formula |
|---|---|
| Ouabain | C₂₉H₄₄O₁₂ |
| Digoxin | C₄₁H₆₄O₁₄ |
| Marinobufagenin (MBG) | C₂₄H₃₂O₅ |
| Oleic acid | C₁₈H₃₄O₂ |
| Docosahexaenoic (DHA) | C₂₂H₃₂O₂ |
| Glutathione disulfide (GSSG) | C₂₀H₃₂N₆O₁₂S₂ |
| Allyl isothiocyanate | C₄H₅NS. |

4. The method of claim 3, wherein the agonist of receptor Na/K-ATPase/Src complex comprises at least two of: ouabain, digoxin, marinobufagenin (MBG), oleic acid, docosahexaenoic (DHA), glutathione disulfide (GSSG), and allyl isothiocyanate.

5. The method of claim 2, wherein the antagonist compound of receptor Na/K-ATPase/Src complex comprises at least one of:

| Antagonist Compound Name | Formula |
|---|---|
| 3,4,5-Trihydroxyxanthone (MB5) | C₁₃H₈O₅ |
| 3,4,5,6-Tetrahydroxyxanthone (MB7) | C₁₃H₈O₆ |
| Curcumin | C₂₁H₂₀O₆ |
| Bisdemethoxycurcumin | C₁₉H₁₆O₄ |
| Tanshinone I | C₁₈H₁₉O₃ |
| Sodium danshensu | C₉H₉O₅Na |
| Astragaloside IV | C₄₁H₆₈O₁₄ |
| Ferulic acid | C₁₀H₁₀O₄ |
| Tanshinone IIA | C₁₉H₁₈O₃. |

6. The method of claim 5, wherein the antagonist of receptor Na/K-ATPase/Src complex comprises two or more of: 3,4,5-trihydroxyxanthone (MB5), 3,4,5,6-tetrahydroxyxanthone (MB7), curcumin, bisdemethoxycurcumin, tanshinone I, sodium danshensu, astragaloside IV, ferulic acid and tanshinone IIA.

7. The method of claim 2, wherein the antagonist of receptor Na/K-ATPase/Src complex, comprises a combination of: tanshinone I, sodium danshensu, astragaloside IV and ferulic acid.

8. The method of claim 2, wherein the agonist of the receptor Na/K-ATPase/Src complex comprises cardiotonic steroids.

9. The method of claim 2, wherein the agonist and/or antagonists of receptor Na/K-ATPase/Src complex is found in one or more of: tan seng, red sage root, radix salvia miltiorrhizae, dang gui, angelica sinensis, huang qi and astragalus.

10. The method of claim 1, wherein the compound is for the treatment of a disorder associated with one or more of: cardiac hypertrophy, tissue fibrosis, congestive heart failure, cancer, wound or skin lesion.

11. The method of claim 1, wherein the compound comprises a mixture of compounds.

12. The method of claim 11, wherein the cell based assay comprises a LLC-PK1 derived α1 knockdown PY-17 cell, a first control cell comprised of P11, and a second control cell comprised of AAC-19.

13. The method of claim 12, wherein the first control cell P11 comprises LLC-PK1transfected with empty vector, and wherein the second control cell AAC-19 comprises rat α1-rescued PY-17 cells.

14. The method of claim 11, wherein the cell based assay comprises a pair of cell lines, LL-A416P-4 and LL-A420P-20, wherein mutation of A420 to P results in inability of expressed Na/K-ATPase to bind and form a functional receptor complex as activated Src in A416P-rescued cells but not A420P mutant-rescued cells.

15. The method of claim 11, wherein the cell based assay comprises cell lines (LY-I279A-3, LY-F286A-19), wherein expressed I279A or F286A mutant Na/K-ATPase is defective in conformational transition.

16. The method of claim 15, wherein I279A and F286A mutants are defective in E1 to E2 and E2 to E1 conformational transition, respectively.

17. An herbal medicinal preparation, comprising effective amounts one or more raw medicinal materials selected from: tan seng, red sage root, radix salvia miltiorrhizae, dang gui, angelica sinensis, huang qi and astragalus,
wherein an agonist or antagonist of receptor Na/K-ATPase/Src complex found in each of the raw medicinal materials is present in an effective amount ranging from about 0.1 nM to about 10 nM.

18. The preparation of claim 17, wherein the agonist comprises one or more of: ouabain, digoxin, marinobufagenin (MBG), oleic acid, docosahexaenoic (DHA), glutathione disulfide (GSSG) and allyl isothiocyanate.

19. The preparation of claim 17, wherein the antagonist comprises one or more of: 3,4,5-trihydroxyxanthone (MB5), 3,4,5,6-tetrahydroxyxanthone (MB7), curcumin, bisdemethoxycurcumin, tanshinone I, sodium danshensu, astragaloside IV, ferulic acid and tanshinone IIA.

20. The preparation of claim 17, wherein the antagonist comprises one or more of: tanshinone I, sodium danshensu, astragaloside IV and ferulic acid.

21. An herbal medicinal preparation for treatment of a disorder associated with one or more of cardiac hypertrophy, tissue fibrosis, congestive heart failure, cancers, wound or skin lesion, comprising the herbal medicinal preparation of claim 17.

22. The herbal medicinal preparation of claim 21, wherein the herbal composition is formulated for oral administration.
